(19) United States
(12) Patent Application Publication
(10) Pub. No.: US 2012/0065674 A1
(43) Pub. Date: Mar. 15, 2012

(54) METHODS AND MATERIALS FOR CLOSING AN OPENING

(76) Inventor: Michael J. Levy, Rochester, MN (US)

(21) Appl. No.: 13/209,869

(22) Filed: Aug. 15, 2011

Related U.S. Application Data
(60) Provisional application No. 61/374,118, filed on Aug. 16, 2010.

(51) Int. Cl. A61B 17/10

(52) U.S. Cl. 606/214

(57) ABSTRACT

This document provides methods and materials for closing an opening (e.g., an incision) within a mammal. For example, methods and materials for closing a transluminal incision created during a natural orifice transluminal endoscopic surgery are provided.

### CROSS-REFERENCE TO RELATED APPLICATIONS

This application claims the benefit of U.S. Provisional Application Ser. No. 61/374,118, filed Aug. 16, 2010. The disclosure of the prior application is considered part of (and is incorporated by reference in) the disclosure of this application.

### BACKGROUND

1. Technical Field

This document relates to methods and materials involved in closing an opening. For example, this document relates to methods and materials that can be performed endoscopically to close an opening (e.g., a translumenal opening) within a mammal.

2. Background Information

Natural orifice transluminal endoscopic surgery (NOTES) is a surgical technique that involves performing a “scarless” operation (e.g., an abdominal operation) by passing an endoscope through a natural orifice (e.g., mouth, urethra, anus, etc.) then through an internal incision in, for example, the esophagus, stomach, vagina, bladder, or colon. This procedure avoids any external incisions or scars.

### SUMMARY

This document provides methods and materials that can be used to close an opening (e.g., an incision) within a mammal. For example, this document provides methods and materials that can be used to close a transluminal incision created during a natural orifice transluminal endoscopic surgery.

In general, one aspect of this document features a device for endoscopically closing an internal opening within a mammal. The device comprises, or consists essentially of, (a) an endoscope having as endoscope lumen, (b) a catheter having a catheter lumen, wherein the catheter is configured to be at least partially housed within the endoscope lumen, (c) an expandable member configured to extend distally from the catheter lumen, and (d) a patch releasably attached to the expandable member, wherein a face of the patch comprises an adhesive or a component of an adhesive configured to adhere the patch to at least some tissue about the opening, thereby closing the opening. The mammal can be a human. The opening can be a translumenal opening. The opening can be formed during a natural orifice transluminal endoscopic surgery. The catheter can be configured to extend beyond the distal end of the endoscope. The expandable member can be a basket, cage, or mesh. The expandable member can be a fan.

Another aspect of this document features a device for endoscopically closing an internal opening within a mammal. The device comprises, or consists essentially of, (a) an endoscope having as endoscope lumen, (b) a catheter having a catheter lumen, wherein the catheter is configured to be at least partially housed within the endoscope lumen, and (c) a balloon configured to extend distally from the catheter lumen, wherein the balloon comprises a pre-defined break region and a patch region, wherein the break region is a region of the balloon configured to break when the balloon is over inflated, and wherein a face of the patch region comprises an adhesive or a component of an adhesive configured to adhere the patch region to at least some tissue about the opening, thereby closing the opening. The mammal can be a human. The opening can be a translumenal opening. The opening can be formed during a natural orifice transluminal endoscopic surgery. The catheter can be configured to extend beyond the distal end of the endoscope. The balloon can be bioabsorbable.

Another aspect of this document features a device for endoscopically closing an internal opening within a mammal. The device comprises, or consists essentially of, (a) an endoscope having as endoscope lumen, (b) a catheter having a catheter lumen, wherein the catheter is configured to be at least partially housed within the endoscope lumen, (c) a delivery member configured to be at least partially housed within the catheter lumen and configured to extend distally from the catheter lumen, and (d) a balloon releasably attached to the delivery member, wherein the balloon comprises a patch region, and wherein a face of the patch region comprises an adhesive or a component of an adhesive configured to adhere the patch region to at least some tissue about the opening, thereby closing the opening. The mammal can be a human. The opening can be a translumenal opening. The opening can be formed during a natural orifice transluminal endoscopic surgery. The catheter can be configured to extend beyond the distal end of the endoscope. The balloon can be bioabsorbable. The balloon can be configured to be released from the delivery member when the balloon is over inflated.

Another aspect of this document features a device for endoscopically closing an internal opening within a mammal. The device comprises, or consists essentially of, (a) an endoscope having as endoscope lumen, (b) a catheter having a catheter lumen, wherein the catheter is configured to be at least partially housed within the endoscope lumen, (c) two or more hook members configured to be at least partially housed within the catheter lumen and configured to extend distally from the catheter lumen, wherein the hook members are configured to attach to tissue about the opening and form a neck-like structure when the hook members are moved, and (d) a banding member configured to be at least partially housed within the catheter lumen or the endoscope lumen, wherein the banding member comprises a deployable band configured to at least partially hold the neck-like structure. The mammal can be a human. The opening can be a translumenal opening. The opening can be formed during a natural orifice transluminal endoscopic surgery. The catheter can be configured to extend beyond the distal end of the endoscope.

Another aspect of this document features a method for endoscopically closing an internal opening within a mammal. The device comprises, or consists essentially of, (a) inserting an endoscope into the mammal to a position proximal to the opening, (b) advancing an expandable member from the distal end of the endoscope toward the opening, wherein the expandable member comprises a releasable patch having an adhesive or a component of an adhesive, (c) adhering the patch to tissue about the opening, thereby closing the opening, and (d) releasing the patch from the expandable member.

Another aspect of this document features a method for endoscopically closing an internal opening within a mammal. The method comprises, or consists essentially of, (a) inserting an endoscope into the mammal to a position proximal to the opening, (b) advancing an expandable balloon from the distal end of the endoscope toward the opening, wherein the balloon comprises a patch comprising an adhesive or a component of an adhesive, (c) adhering the patch region to tissue about the opening, thereby closing the opening, and (d) over-inflating the expandable balloon to break the expandable balloon. The expandable balloon can comprise a pre-defined break region, and the expandable balloon can break along the predefined break region during the over-inflating step (d).

Another aspect of this document features a method for endoscopically closing an internal opening within a mammal. The method comprises, or consists essentially of, (a) inserting an endoscope into the mammal to a position proximal to the opening, (b) advancing an delivery member comprising a releasable, expandable balloon from the distal end of the endoscope toward the opening, wherein the balloon comprises a patch comprising an adhesive or a component of an adhesive, (c) adhering the patch region to tissue about the opening, thereby closing the opening, and (d) releasing the balloon from the delivery member.

Another aspect of this document features a method for endoscopically closing an internal opening within a mammal. The method comprises, or consists essentially of, (a) inserting an endoscope into the mammal to a position proximal to the opening, (b) advancing two or more hook members from the distal end of the endoscope into tissue about the opening, (c) retracting or advancing the two or more hook members to form a neck-like structure with the tissue, and (d) positioning a band at least partially about the neck-like structure. The method can comprise positioning two or more bands at least partially about the neck-like structure.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A and 1B are side views of a device having an expandable member for deploying a patch to an internal opening, in accordance to one embodiment provided herein. FIG. 1A is a view showing the deployment of a patch to the proximal face of the internal opening, in accordance to one embodiment provided herein. FIG. 1B is a view showing the deployment of a patch to the distal face of the internal opening, in accordance to one embodiment provided herein.

FIG. 2 is a side view of a device having an expandable balloon for deploying a patch to an internal opening, in accordance to one embodiment provided herein.

FIG. 3 is a side view of a device having an expandable balloon for deploying a patch to an internal opening, in accordance to one embodiment provided herein.

FIG. 4 is a side view of a device having an expandable fan member for deploying a patch to an internal opening, in accordance to one embodiment provided herein.

FIG. 5 is a side view of a multiple expandable members for deploying a patch to an internal opening, in accordance to one embodiment provided herein.

FIGS. 6A-D are side views of a device having retractable hook members and a banding member for banding an internal opening, in accordance to one embodiment provided herein. FIG. 6A is a view showing the deployment of hook members to prepare the internal opening for banding, in accordance to one embodiment provided herein. FIG. 6B is a view showing the placement of bands around the prepared tissue (e.g., tissue neck), in accordance to one embodiment provided herein. FIG. 6C is a view showing the advancement of the hook members in preparation for removal, in accordance to one embodiment provided herein. FIG. 6D is a view showing the removal of the hook members, in accordance to one embodiment provided herein.

FIG. 7 is a side view of banded tissue that can be cauterized, in accordance to one embodiment provided herein.

FIGS. 8A-F is a side view of hook member configurations, in accordance to exemplary embodiments provided herein.

FIGS. 9A-D are side views of a device having retractable hook members with one or more sutures for closing an internal opening, in accordance to one embodiment provided herein. FIG. 9A is a view showing the advancement of a device towards an opening, in accordance to one embodiment provided herein. FIG. 9B is a view showing the deployment of hook members to prepare the internal opening for closure, in accordance to one embodiment provided herein. FIG. 9C is a view showing suturing to close the opening, in accordance to one embodiment provided herein. FIG. 9D is a view showing another example of suturing to close the opening, in accordance to one embodiment provided herein.

FIG. 10 is a side of a device being used to deliver a patch to the pylorus of a stomach, in accordance to one embodiment provided herein.

### DETAILED DESCRIPTION

This document provides methods and materials that can be used to close an opening (e.g., an incision) within a mammal. For example, this document provides methods and materials that can be used to close a transluminal incision created during a natural orifice transluminal endoscopic surgery.

In some cases, a device provided herein can include an expandable member configured to deploy a patch, a banding member configured to deploy bands, or both to an internal opening within a mammal. Such internal openings can be located within any type of tissue within a mammal (e.g., a human, horse, cow, dog, or cat). For example, an internal opening can be an incision that is created within luminal tissue during a natural orifice transluminal endoscopic surgery. Examples of such tissues include, without limitation, esophagus, stomach, vagina, bladder, colon, duodenal, gall bladder, and urethral tissue. An opening can range in size from about 1 mm up to about 4 to 5 cm (e.g., 1 mm, 5 mm, 10 mm, 25 mm, 50 mm, 75 mm, 1 cm, 1.25 cm, 1.5 cm, 1.75 cm, 2 cm, 2.5 cm, 3 cm, 3.5 cm, 4 cm, 4.5 cm, or 5 cm). For example, an opening can range in size from about 1 mm to about 5 cm (e.g., from about 1 mm to about 5 cm, from about 5 mm to about 5 cm, from about 10 mm to about 5 cm, from about 25 mm to about 5 cm, from about 50 mm to about 5 cm, from about 75 mm to about 5 cm, from about 1 cm to about 5 cm, from about 1.25 cm to about 5 cm, from about 1.5 cm to about 5 cm, from about 1.75 mm to about 5 cm, from about 2 mm to about 5 cm, from about 1 mm to about 4.5 cm, from about 1 mm to about 4 cm, from about 1 mm to about 3.5 cm, from about 1 mm to about 3 cm, from about 1 mm to about 2.5 cm, from about 1 mm to about 2 cm, from about 1 mm to about 1.75 cm, from about 1 mm to about 1.5 cm, from about 5 mm to about 2 cm, from about 10 mm to about 2 cm, from about 5 mm to about 1.5 cm, or from about 10 mm to about 1.5 cm).

In some cases, a device provided herein can be used to close partially or completely a natural opening within a mammal. For example, a device provided herein can be used to deploy a patch having a defined opening to the pylorus of a mammal's stomach. In this case, the size of the opening within the patch can control the flow of food material from the mammal's stomach to the duodenum. Other examples of natural openings within a mammal that can be partially or completely closed using the devices provided herein include, without limitation, elements of the gastrointestinal lumen, biliary system, urinary system, and vascular system.

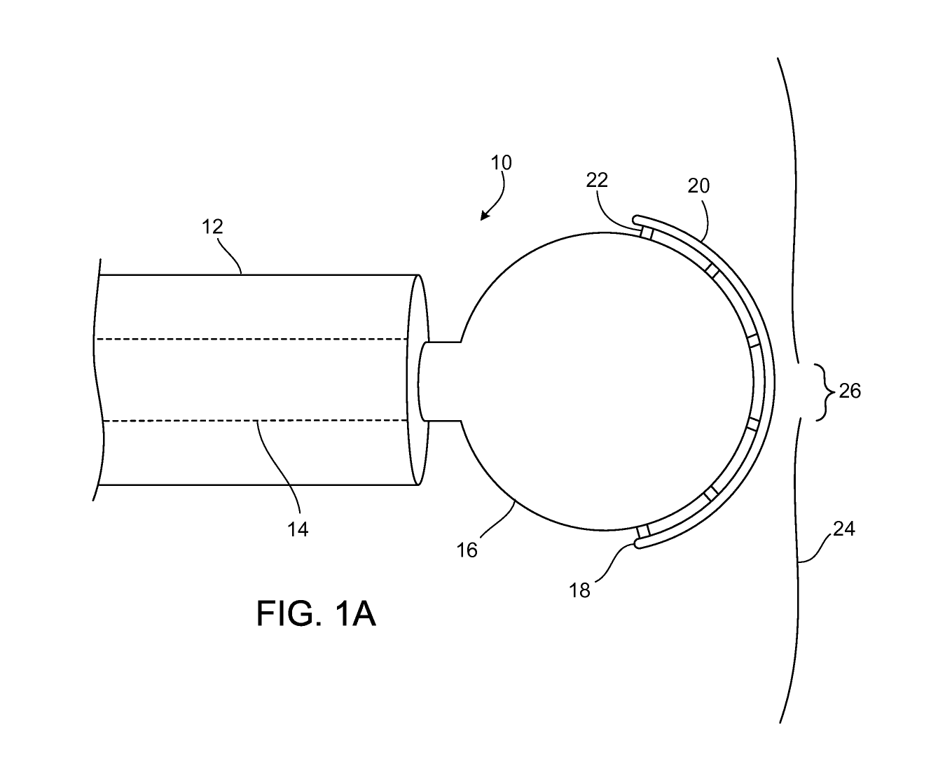

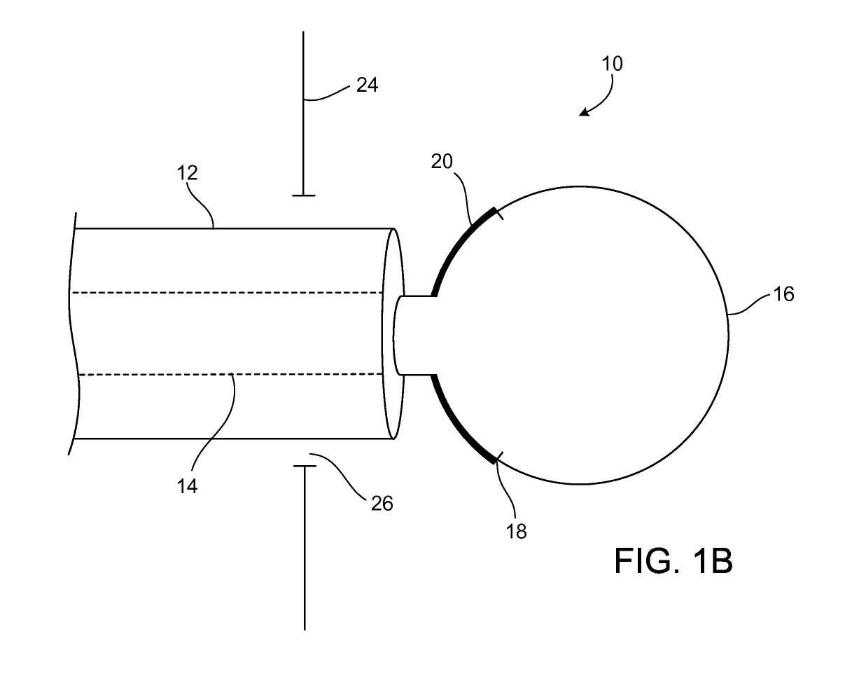

With respect to FIG. 1A, device 10 includes endoscope 12 and catheter 14. Endoscope 12 can be any appropriate type of endoscope including, without limitation, gastrointestinal, cystoscopic, laparoscopic, and arthroscopic endoscopes. Catheter 14 can be any appropriate type of catheter or sheath configured to function within endoscope 12 and to allow advancement of expandable member 16 beyond the distal end of catheter 14. In some cases, catheter 14 and expandable member 16 can move independently or be integrated as one piece. Expandable member 16 can be configured as a cage, mesh, or basket.

The distal end of expandable member 16, when expanded, includes patch 18. Patch 18 can be any appropriate type of patch. For example, a patch or membrane can be inert or bioabsorbable. Examples of materials that can be used to make a patch provided herein include, without limitation, silicone, rubber, plastic, polymers, shape-memory materials (e.g., nitinol), collagen, and bovine pericardium. The distal face of patch 18 can include adhesive 20, and the proximal face of patch 18 can include one or more release members 22. Adhesive 20 can be any appropriate adhesive material such as a ready-to-use adhesive material, an activatable adhesive, or one component of a multi-component adhesive. In some case, adhesive 20 can be one component of a two component adhesive. Examples of ready-to-use adhesive materials include, without limitation, fibrin glue, cyanoacrylate glues, and oxidized regenerated cellulose. Examples of activatable adhesive materials include, without limitation, magnetic or electromagnetic devices. Examples of multi-component adhesive materials include, without limitation, cyanoacrylates. Release members 22 can be weaker glue than that used for the patch itself, sutures, mechanically actuatable hooks, twist-off mechanisms, weak magnetic attraction, locking devices, or cautery devices.

During use, endoscope 12 can house catheter 14 and be advanced towards tissue 24 having opening 26. Once in position, expandable member 16 can be advanced beyond the distal end of endoscope 12 and the distal end of catheter 14. Once expanded, the expandable member 16 can be positioned to place the distal face of patch 18 into contact with the tissue of tissue 24 that surrounds opening 26. Adhesive 20 can attach patch 18 to the tissue, and release members 22 can allow patch 18 to be separated from expandable member 16. When one component of a multiple component adhesive is used as adhesive 20 and patch 18 is positioned over opening 26 within tissue 24, the other component(s) can be delivered to adhesive 20 via catheter 14 or a separate catheter within endoscope 12. In some cases, the delivered adhesive components can be allowed to diffuse through patch 18 to make contact with adhesive 20, thereby securing patch 18 to the tissue of tissue 24 that surrounds opening 26.

In some cases, device 10 can be used to deploy patch 18 using a pull-back method (FIG. 1B). With reference to FIG. 1B, device 10 can include patch 18 having adhesive 20 on the proximal face of patch 18. In such cases, the patch can be positioned on the distal face of the tissue of tissue 24 that surrounds opening 26.

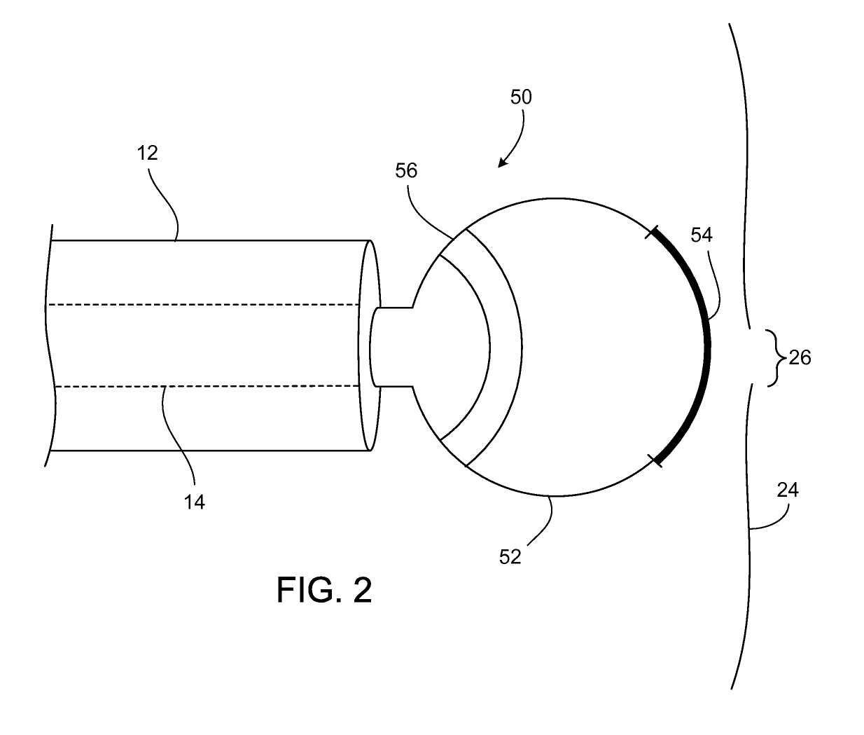

With reference to FIG. 2, device 50 includes endoscope 12 and catheter 14. Catheter 14 can be configured to allow advancement, beyond the distal end of catheter 14, of an expandable member in the form of balloon 52. Balloon 52 can be configured to be a breakable balloon along break region 56. Break region 56 can be a thin section as compared to the other regions of balloon 52. The distal end of balloon 52, when expanded, includes patch 54. Patch 54 can be any appropriate type of patch. For example, a patch or membrane can be inert or bioabsorbable. Examples of materials that can be used to make a patch provided herein include, without limitation, silicone, rubber, plastic, polymers, shape-memory materials (e.g., nitinol), collagen, and bovine pericardium. In some cases, patch 54 can be the same material used to make balloon 52. The distal face of patch 54 can include an adhesive as described herein.

During use, endoscope 12 can house catheter 14 and can be advanced towards tissue 24 having opening 26. Once in position, balloon 52 can be advanced beyond the distal end of endoscope 12 and the distal end of catheter 14. Once expanded, balloon 52 can be positioned to place the distal face of patch 54 into contact with the tissue of tissue 24 that surrounds opening 26. An adhesive can attach patch 54 to the tissue, and balloon 52 can be inflated to break along break region 56, thereby separating patch 54 from the main part of the device.

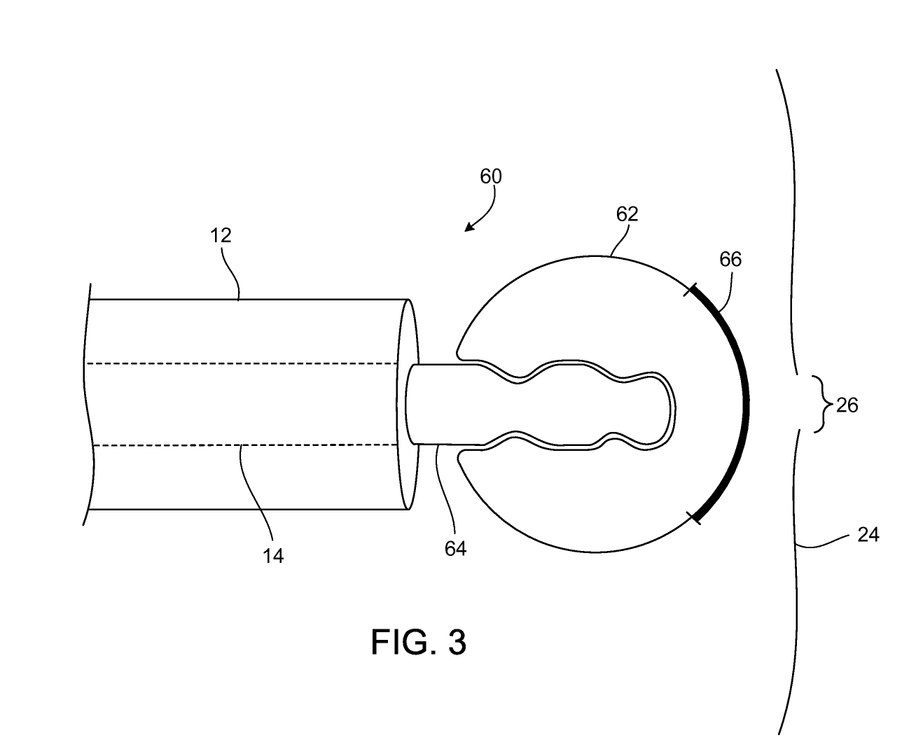

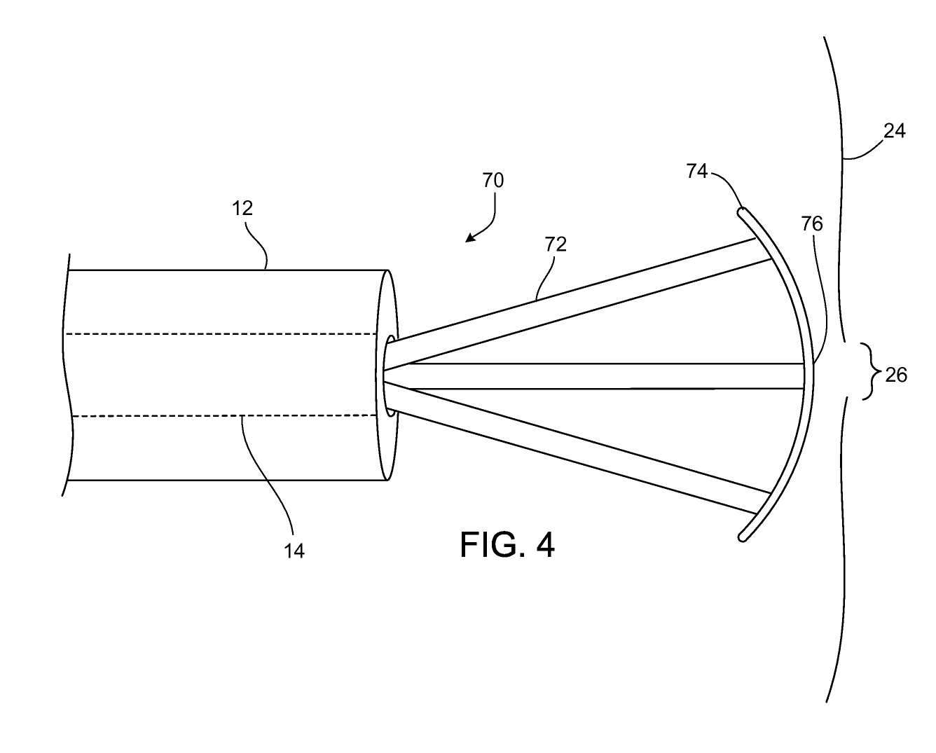

With reference to FIG. 3, device 60 includes endoscope 12 and catheter 14. Catheter 14 can be configured to allow advancement, beyond the distal end of catheter 14, of an expandable member in the form of balloon 62 releasably engaged to delivery member 64 via, for example, notches, magnets, or screws. Once overinflated, balloon 62 can be configured to be released from delivery member 64. The distal end of balloon 62, when expanded, includes patch 66, and the distal face of patch 66 can include an adhesive as described herein.

During use, endoscope 12 can house catheter 14 and can be advanced towards tissue 24 having opening 26. Once in position, balloon 62 can be advanced beyond the distal end of endoscope 12 and the distal end of catheter 14. Once expanded, balloon 62 can be positioned to place the distal face of patch 66 into contact with the tissue of tissue 24 that surrounds opening 26. An adhesive can attach patch 66 to the tissue, and balloon 62 can be inflated to a point such that balloon 62 is released from delivery member 64. Once release, balloon 62 can deflate and remain at the delivery site.

With reference to FIG. 4, device 70 includes endoscope 12 and catheter 14. Catheter 14 can be configured to allow advancement, beyond the distal end of catheter 14, of an expandable member in the form of a fan member 72. The distal end of fan member 72, when expanded, includes patch 74. Patch 74 can be any appropriate type of patch. For example, a patch or membrane can be inert or bioabsorbable. The distal face of patch 74 can include adhesive 76 as described herein, and the proximal face of patch 74 can include one or more release members.

During use, endoscope 12 can house catheter 14 and can be advanced towards tissue 24 having opening 26. Once in position, fan member 72 can be advanced beyond the distal end of endoscope 12 and the distal end of catheter 14. Once expanded, fan member 72 can be positioned to place the distal face of patch 74 into contact with the tissue of tissue 24 that surrounds opening 26. Adhesive 76 can attach patch 74 to the tissue, and the release members can allow patch 74 to be separated from fan member 72.

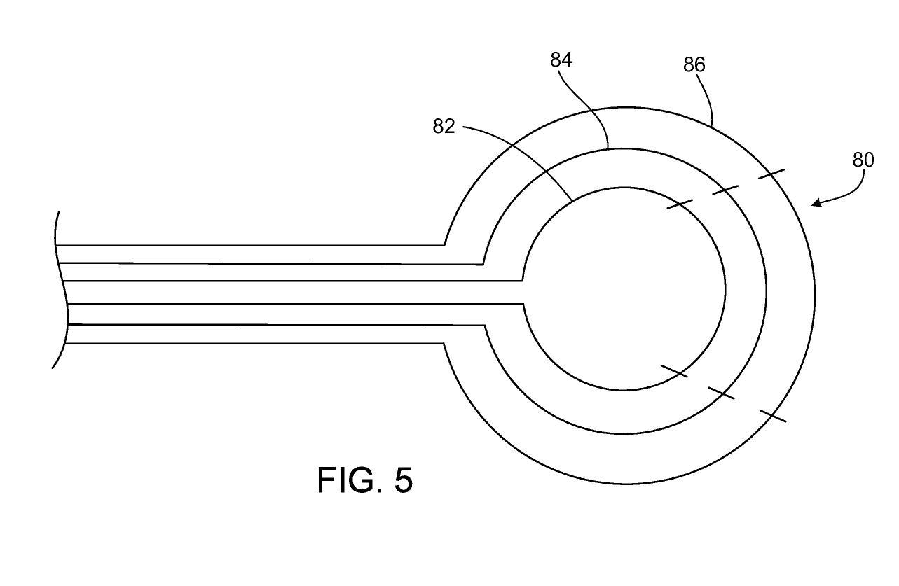

In some cases, a device provided herein can be configured to provide multiple patches. For example, as shown in FIG. 5, a series of embedded expandable members (e.g., balloons) can be loaded into a single catheter for delivery to multiple openings within a mammal. With reference to FIG. 5, an outer expandable member 86 can be located outside of inner expandable member 84, which can be located outside of inner expandable member 82.

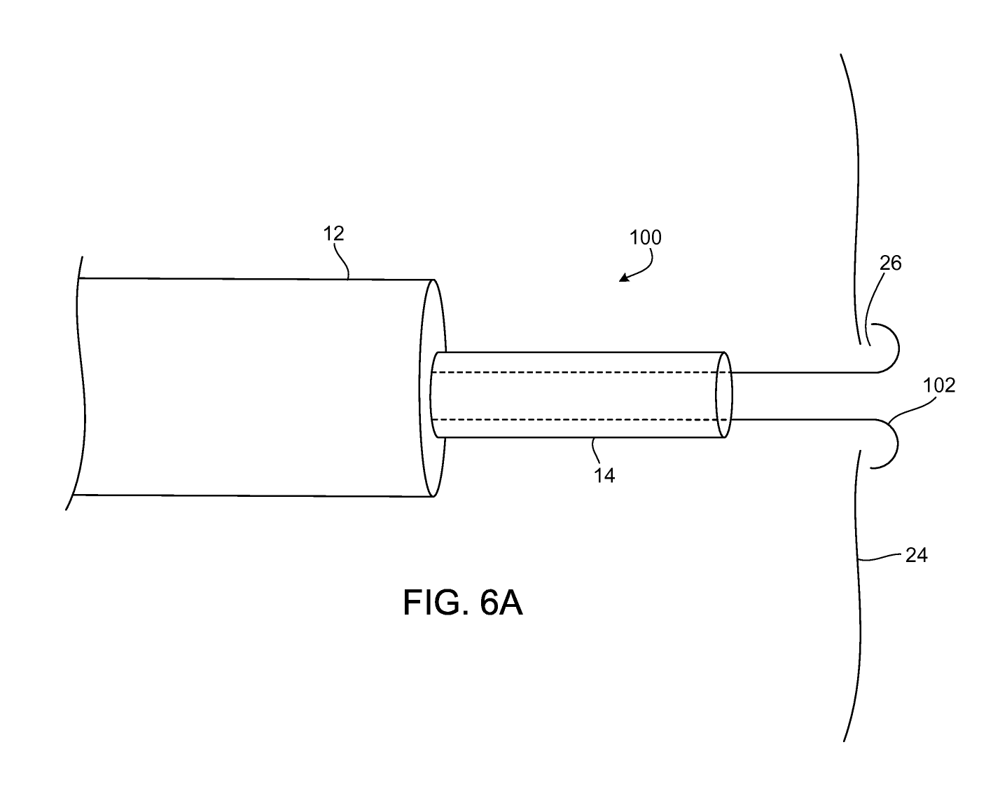

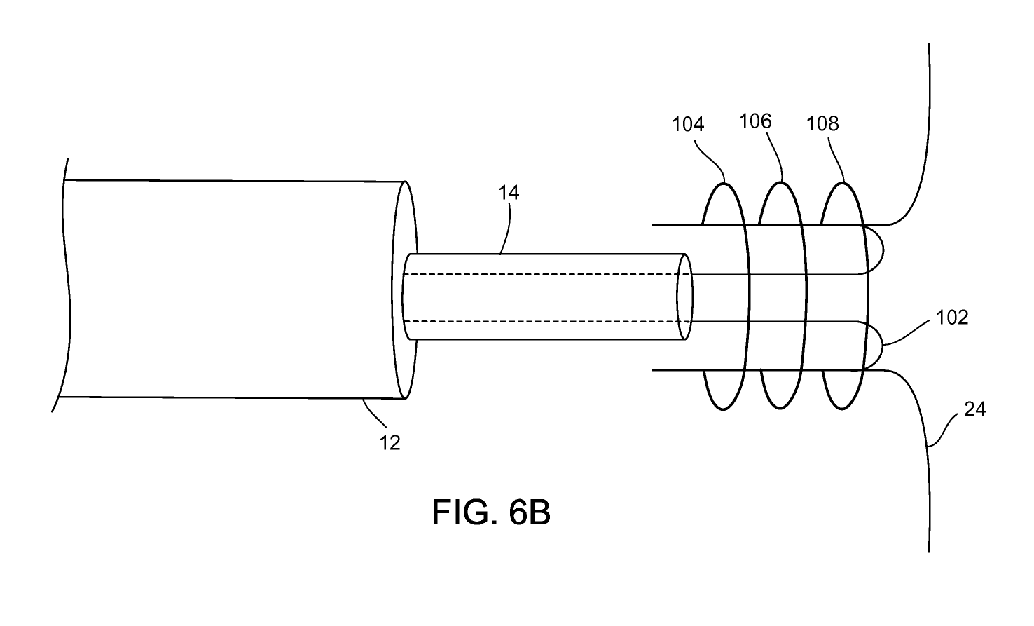

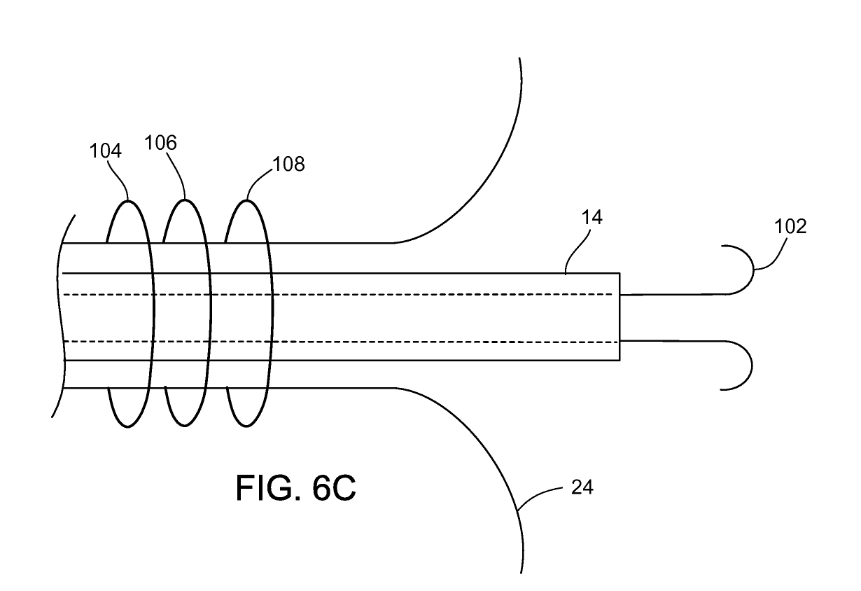

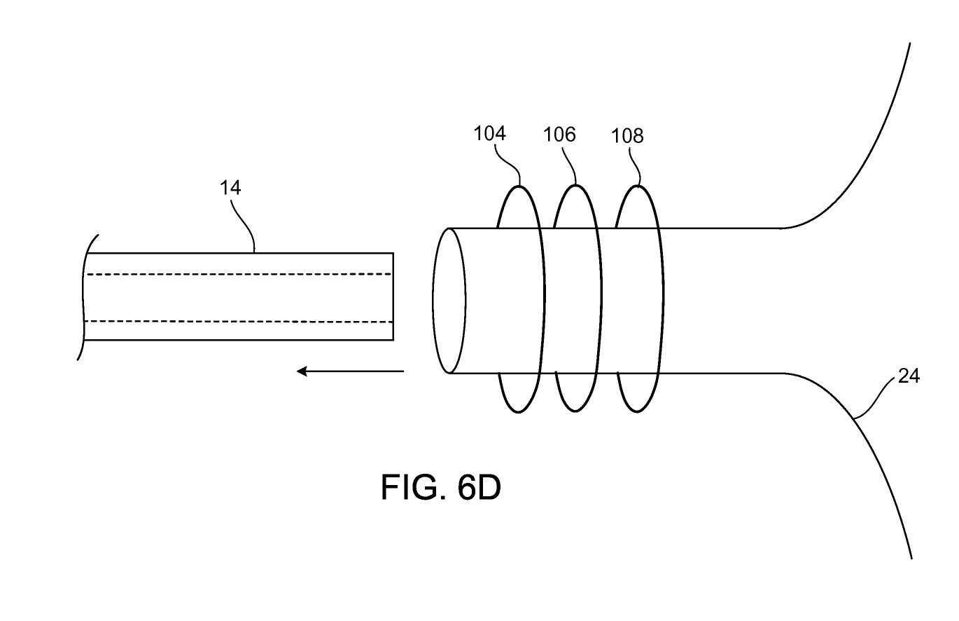

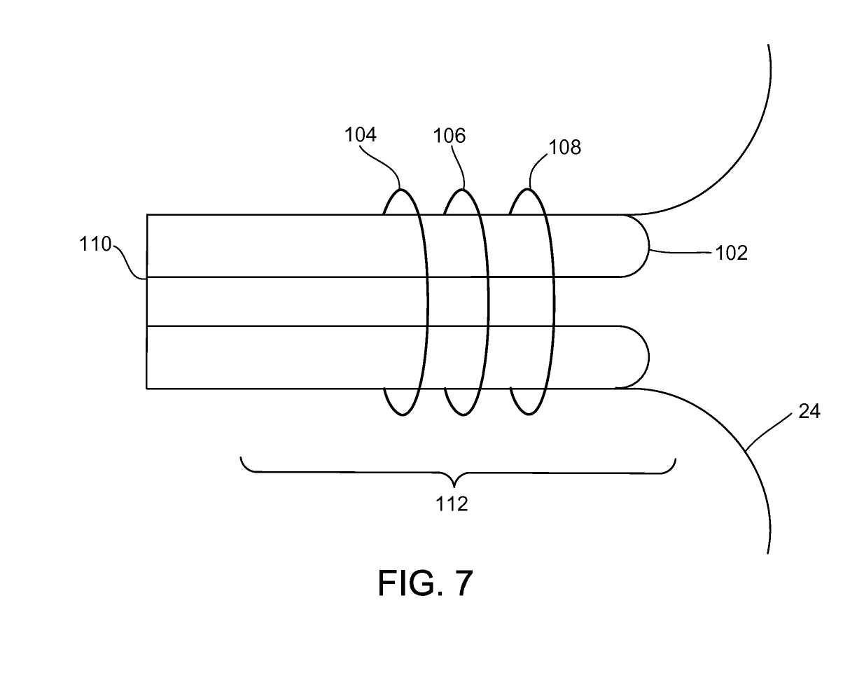

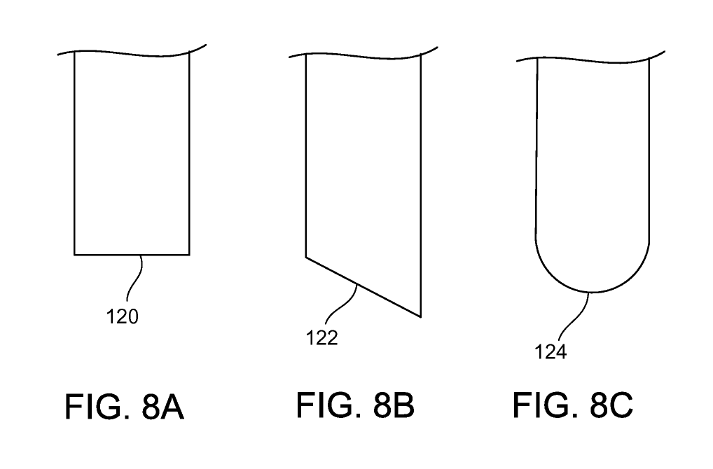

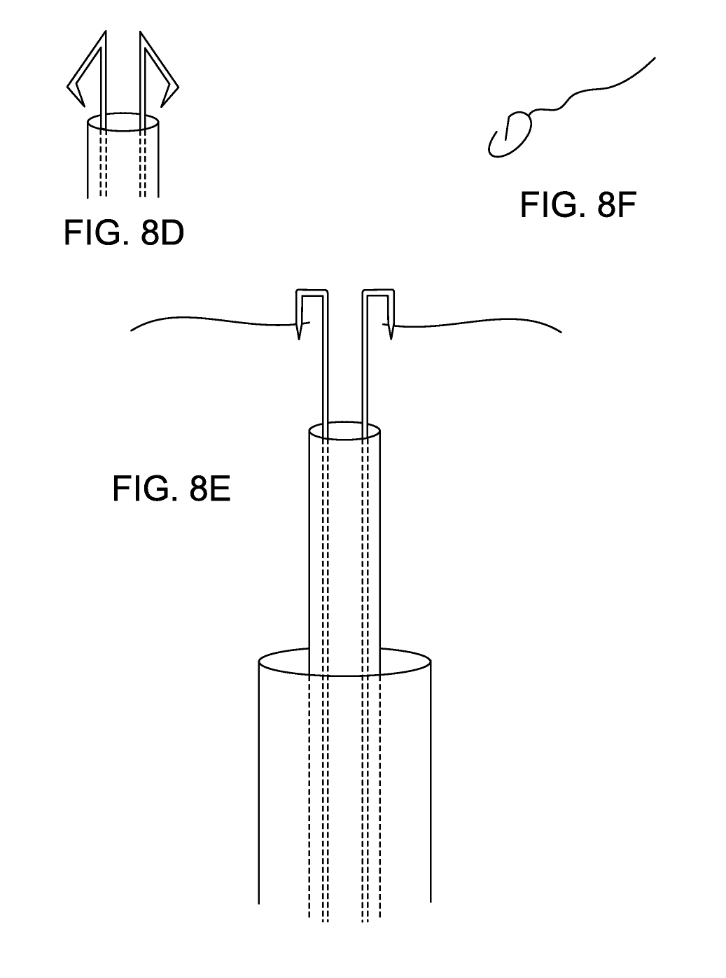

With reference to FIG. 6A, device 100 includes endoscope 12 and catheter 14. Endoscope 12 can be any appropriate type of endoscope including, without limitation, gastrointestinal, cystoscopic, laparoscopic, and arthroscopic endoscopes. Catheter 14 can be any appropriate type of catheter or sheath configured to function within endoscope 12 and to allow advancement of catheter 14 beyond the distal end of endoscope 12. Catheter 14 can be configured to allow hook members 102 to extend beyond the distal end of catheter 14. Hook members 102 are configured to extend into tissue surrounding opening 26 such that the tissue can be pulled toward endoscope 12, thereby forming a neck-like configuration. Any number of hook members can be used. For example, two, three, four, five, six, seven, eight, or more hook members can be used. In some cases, a hook member can have a hook region that extends between about 2 to 3 mm away from the shank region of the hook member. As shown in FIGS. 8A-F, hook members 102 can have any appropriate shape. Banding members 104, 106, and 108 can be applied from catheter 14 or a separate catheter (or a separate device) over the neck-like configuration to hold the tissue in place, for example, as shown in FIG. 6B. Banding members 104, 106, and 108 can be made of any appropriate material including, without limitation, rubber, plastics, and polymers. In some cases, a cap or band device such as those described elsewhere (e.g., U.S. Patent Application Publication Nos. 2009/0182198 and 2008/0242932 and U.S. Pat. No. 7,641,652) can be used as a banding member. Once the bands are applied, hook members 102 can be removed from the tissue by advancing catheter 14 and hook members 102 forward, for example, as shown in FIG. 6C. Once hook members 102 are removed from the tissue, hook members 102 can be positioned within catheter 14 and withdrawn from the mammal, for example, as shown in FIG. 6D.

In some cases, hook members 102 can be detached from the device and left within the mammal (FIG. 7). In some cases, proximal tissue edge 110, neck tissue region 112, or both can be cauterized, glued, stapled, sutured, etc.

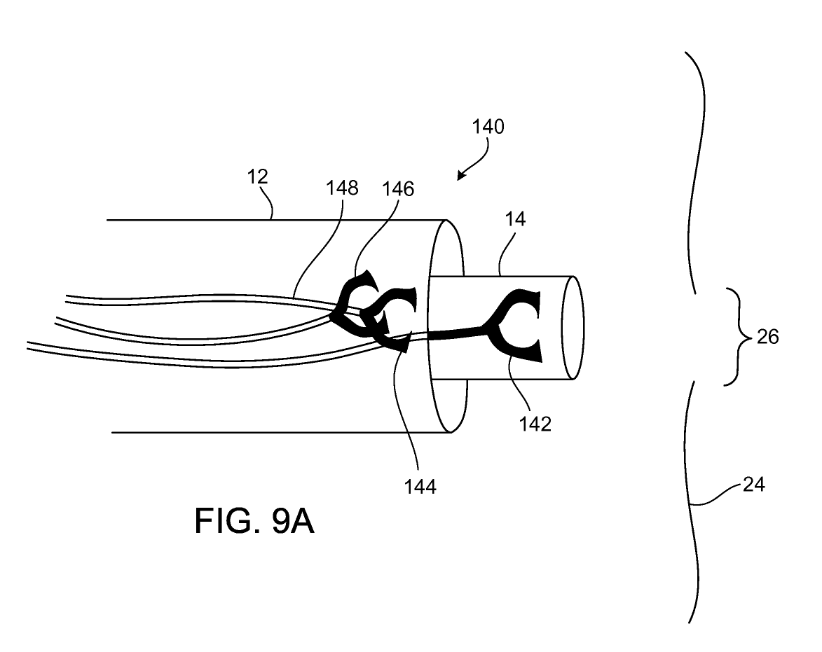

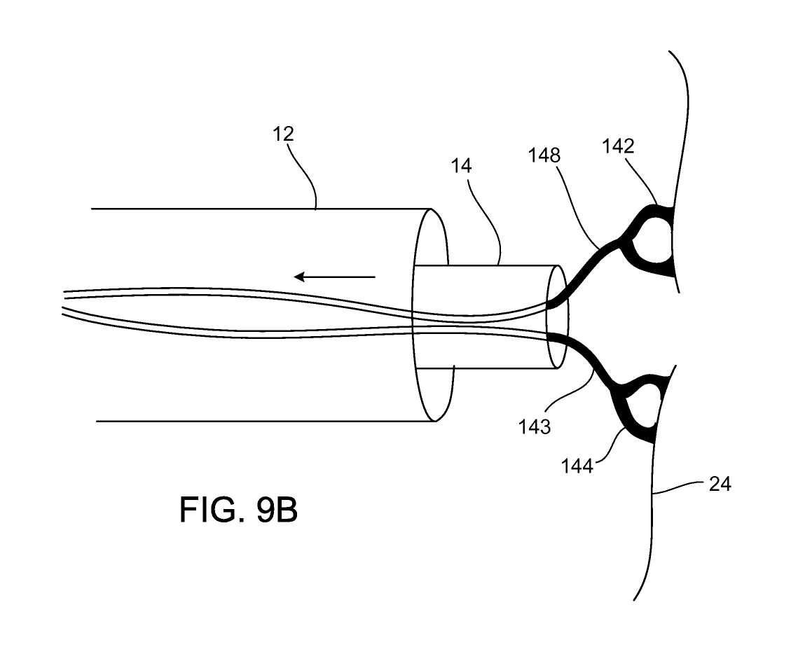

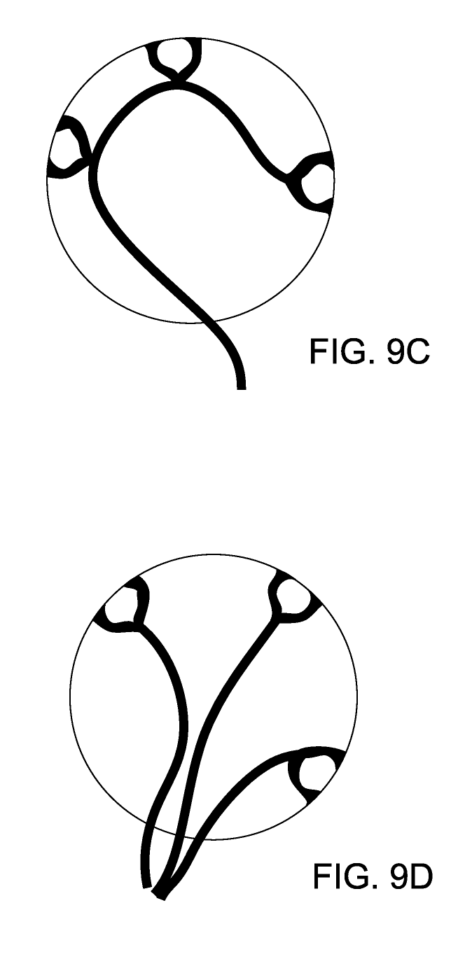

With reference to FIG. 9A, device 140 includes endoscope 12 and catheter 14. Endoscope 12 can be any appropriate type of endoscope including, without limitation, gastrointestinal, cystoscopic, laparoscopic, and arthroscopic endoscopes. Catheter 14 can be any appropriate type of catheter or sheath configured to function within endoscope 12 and to allow advancement of catheter 14 beyond the distal end of endoscope 12. Catheter 14 can be configured to allow one or more clip members 142, 144, and 146 to extend beyond the distal end of catheter 14. Clip members 142, 144, and 146 can be configured to include one or more sutures and can be configured to extend into tissue surrounding opening 26 such that the tissue can be pulled together to close opening 26. Like hook members 102, clips 142, 144, and 146 can have any appropriate shape (FIGS. 8A-F). In some cases, clips 142, 144, and 146 can be configured as endoclips and/or can be configured to have a pointed tip. In some cases, the clips can be configured to be a lock and key type version of an endoclip. In such cases, one tip can be the lock, and the other can be the key. Such a configuration can provide additional security in keeping it closed. Once the clips are applied to the tissue (FIG. 9B), a single suture (FIG. 9C) or multiple sutures (FIG. 9D) can be used to close opening 26.

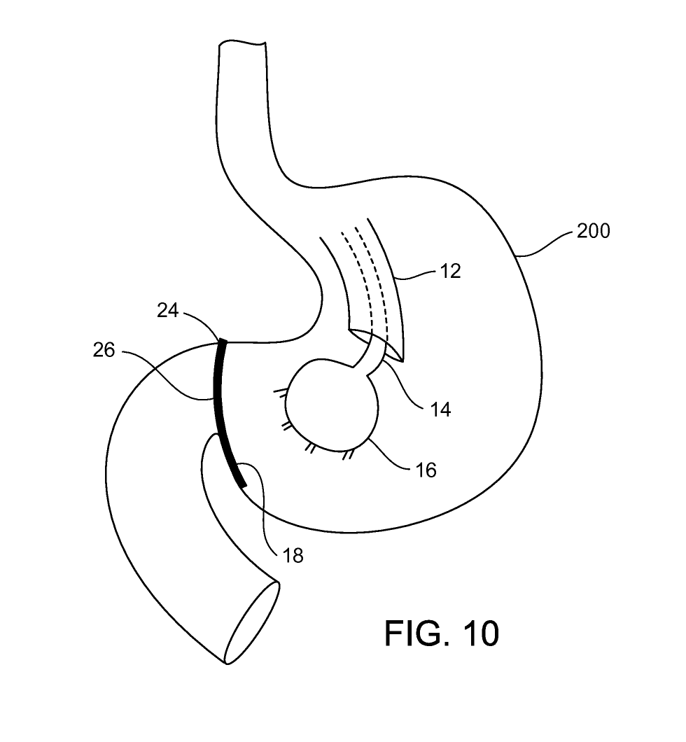

With reference to FIG. 10, a device provided herein can be used to deploy patch 18 to an opening 26 (e.g., pylorus) of a mammal's stomach 200. In such cases, patch 18 can be configured to include a defined opening to allow food material to pass from stomach 200 to the duodenum or can be cauterized or otherwise manipulated to create an appropriate opening. For example, after deploying patch 18, a cauterizing device can be used to generate an opening or multiple openings in patch 18 of appropriate size to control or reduce the flow of food from stomach 200 to the duodenum. Such an opening or openings can range from about 0.5 mm to about 5 mm (e.g., about 0.5 mm to about 4 mm, about 0.5 mm to about 3 mm, about 0.5 mm to about 2 mm, about 0.5 mm to about 1 mm, about 1 mm to about 4 mm, about 1 mm to about 3 mm, about 1 mm to about 2 mm, about 1.5 mm to about 4 mm, or about 1.5 mm to about 3 mm).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for endoscopically closing an internal opening within a mammal, wherein said method comprises:
(a) inserting an endoscope into said mammal to a position proximal to said opening,
(b) advancing an expandable member from the distal end of said endoscope toward said opening, wherein said expandable member comprises a releasable patch having an adhesive or a component of an adhesive,
(c) adhering said patch to tissue about said opening, thereby closing said opening, and
(d) releasing said patch from said expandable member.

2. The method of claim 1, wherein said mammal is a human.

3. The method of claim 1, wherein said opening is a translumenal opening.

4. The method of claim 1, wherein said opening was formed during a natural orifice transluminal endoscopic surgery.

5. A method for endoscopically closing an internal opening within a mammal, wherein said method comprises:
(a) inserting an endoscope into said mammal to a position proximal to said opening,
(b) advancing an expandable balloon from the distal end of said endoscope toward said opening, wherein said balloon comprises a patch comprising an adhesive or a component of an adhesive,
(c) adhering said patch region to tissue about said opening, thereby closing said opening, and
(d) over-inflating said expandable balloon to break said expandable balloon.

6. The method of claim 5, wherein said mammal is a human.

7. The method of claim 5, wherein said opening is a translumenal opening.

8. The method of claim 5, wherein said opening was formed during a natural orifice transluminal endoscopic surgery.

9. The method of claim 5, wherein said expandable balloon comprises a pre-defined break region, and said expandable balloon breaks along said predefined break region during said over-inflating step (d).

10. A method for endoscopically closing an internal opening within a mammal, wherein said method comprises:
(a) inserting an endoscope into said mammal to a position proximal to said opening,
(b) advancing an delivery member comprising a releasable, expandable balloon from the distal end of said endoscope toward said opening, wherein said balloon comprises a patch comprising an adhesive or a component of an adhesive,
(c) adhering said patch region to tissue about said opening, thereby closing said opening, and
(d) releasing said balloon from said delivery member.

11. The method of claim 10, wherein said mammal is a human.

12. The method of claim 10, wherein said opening is a translumenal opening.

13. The method of claim 10, wherein said opening was formed during a natural orifice transluminal endoscopic surgery.
